(19) United States
(12) Patent Application Publication
(10) Pub. No.: US 2005/0136416 A1
(43) Pub. Date: Jun. 23, 2005

(54) Patient specific array-based assays

(76) Inventors: LauraKay Bruhn, Mountain View, CA (US); Michael T. Barrett, Mountain View, CA (US); Paul K. Wolber, Los Altos, CA (US); Leslie A. Leonard, Portola Valley, CA (US); Richard J. Pittaro, San Carlos, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/747,004

(22) Filed: Dec. 23, 2003

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

Patient specific array-based assays are provided. A feature of the subject patient specific array-based assays is that they include a step of interrogating a set of array features that have been previously identified by a general array-based assay to be relevant to a specific disease and condition of the subject being assayed. Also provided are methods of producing the subject patient specific array-based assays, as well as compositions and kits for use in practicing the subject assays.

### FIELD OF THE INVENTION

The present invention relates to biopolymeric arrays, particularly as employed in clinical assay applications.

### BACKGROUND OF THE INVENTION

Array assays between surface bound binding agents or probes and target molecules in solution may be used to detect the presence of particular biopolymeric analytes in the solution. The surface-bound probes may be oligonucleotides, peptides, polypeptides, proteins, antibodies or other molecules capable of binding with target biomolecules in the solution.

One typical array assay method uses biopolymeric probes immobilized in an array on a substrate such as a glass substrate or the like. A solution containing target molecules (“targets”) that bind with the attached probes is placed in contact with the bound probes under conditions sufficient to promote binding of targets in the solution to the complementary probes on the substrate to form a binding complex that is bound to the surface of the substrate. The binding by target molecules to probe features or spots on the substrate produces a pattern, i.e., a binding complex pattern, on the surface of the substrate that is detected. This detection of binding complexes provides desired information (qualitatively and/or quantitatively) about the target biomolecules in the solution.

The binding complexes may be detected by reading or scanning the array with, for example, optical means, although other methods may also be used, as appropriate for the particular assay. For example, laser light may be used to excite fluorescent labels attached to the targets, generating a signal only in those spots on the array that have a labeled target molecule bound to a probe molecule. This pattern may then be digitally scanned for computer analysis. Such patterns can be used to generate data for biological assays such as the identification of drug targets, single-nucleotide polymorphism mapping, monitoring samples from patients to track their response to treatment, assessing the efficacy of new treatments, etc.

Such array assays find use in a variety of different fields, e.g., genomics (in sequencing by hybridization, SNP detection, differential gene expression analysis, identification of novel genes, gene mapping, finger printing, etc.) and proteomics.

One particular area in which such array-based assays are finding increasing use is in clinical assays, e.g., in which the array assays are performed in a clinical setting to diagnose and/or monitor the progression of a condition in a patient, e.g., a disease condition.

As the use of array-based protocols in clinical applications becomes increasingly popular, there is continued interest in the development of refined array-based protocols that are particularly suited for use in the clinical setting.

Relevant Literature

Representative references that disclose array-based clinical assays include: WO 02/056030; WO 02/084249; WO 02/33415; WO 02/39120; U.S. Pat. Nos. 6,210,878; and 6,171,793.

### SUMMARY OF THE INVENTION

Patient specific array-based assays are provided. A feature of the subject patient specific array-based assays is that they include a step of interrogating a set of array features that have been previously identified by a general array-based assay of the patient to be relevant to a specific disease and condition of the subject being assayed. Also provided are methods of producing the subject array-based assays, as well as compositions and kits for use in practicing the subject assays.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

A “biopolymec” is a polymer of one or more types of repeating units. Biopolymers are typically found in biological systems and particularly include polysaccharides (such as carbohydrates), peptides (which term is used to include polypeptides and proteins) and nucleic acids, as well as their analogs such as those compounds composed of or containing amino acid analogs or non-amino acid groups, or nucleotide analogs or non-nucleotide groups.

A “biomonomer” references a single unit, which can be linked with the same or other biomonomers to form a biopolymer (e.g., a single amino acid or nucleotide with two linking groups one or both of which may have removable protecting groups).

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to about 200 nucleotides in length, such as from about 10 to about 100 nucleotides.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The term “array” encompasses the term “microarray” and refers to an ordered array presented for binding to targets in solution, e.g., nucleic acids, peptides and the like.

An “array,” includes any one-dimensional, two-dimensional or substantially two-dimensional (as well as a three-dimensional) arrangement of addressable regions bearing biopolymers, e.g., nucleic acids, peptides, and the like. Where the arrays are arrays of nucleic acids, the nucleic acids may be adsorbed, physisorbed, chemisorbed, photo-induced cross-linked, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more than ten thousand features, or even more than one hundred thousand features, in an area of less than 20 cm² or even less than 10 cm², e.g., less than about 5 cm², including less than about 1 cm², less than about 1 mm², e.g., 100μ², or even smaller. For example, features may have widths (that is, diameter, for a round spot) in the range from 1 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm.

Array substrates may be flexible (such as a flexible web). When the substrates are flexible, they may be of various lengths including at least 1 m, at least 2 m, or at least 5 m (or even at least 10 m). “Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm. Array substrates may also be reflective and have little or no transparency. The reflectivity may reduce the absorption of the incident illuminating laser light and subsequent heating if the focused laser beam travels too slowly over a region. The substrate may be at least 20% reflective, preferably at least 50% reflective.

Arrays can be fabricated using drop deposition from pulse-jets of either probe precursors, e.g., nucleic acid precursor units (such as monomers), in the case of in situ fabrication, or previously obtained probes, e.g., a previously produced nucleic acid. Such methods are described in detail in, for example, the previously cited references including U.S. Pat. No. 6,242,266, U.S. Pat. No. 6,232,072, U.S. Pat. No. 6,180,351, U.S. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array will detect a particular probe sequence. Array features are typically, but need not be, separated by intervening spaces. In the case of an array in the context of certain embodiments of the present application, the “target” will be referenced as a moiety in a mobile phase (typically fluid), to be detected by “probes” which are bound to the substrate at the various regions. However, in certain embodiments, e.g., Comparative Genomic Hybridization (CGH) applications, it may be more appropriate to view the probes as being in solution and the targets being immobilized on the substrate surface.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

By “remote location,” it is meant a location other than the location at which the array is present and hybridization occurs. For example, a remote location could be another location (e.g., office, lab, etc.) in the same building, city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electronic signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, words such as “top,” “upper,” and “lower” are used in a relative sense only.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

By “clinical assay” is meant an assay or test that is performed on a sample obtained from an individual or patient (also referred to herein as host or subject) in order to provide information on current or future health or condition, diagnosis, treatment, prevention, and/or monitoring of a condition of the individual or patient.

### DETAILED DESCRIPTION OF THE INVENTION

Patient specific array-based assays are provided. A feature of the subject patient specific array-based assays is that they include a step of interrogating a set of array features that have been previously identified by a general array-based assay of the patient to be relevant to a specific disease and condition of the subject being assayed. Also provided are methods of producing the subject array-based assays, as well as compositions and kits for use in practicing the subject assays.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications that might be used in connection with the presently described invention.

In further describing the invention in greater detail than provided in the Summary and as informed by the Background and Definitions provided above, representative embodiments of the subject array-based clinical assays are described first in greater detail, followed by a discussion of representative application in which the subject array-based clinical assays find use, as well as kits and systems that find use in practicing the subject assays.

Methods

As summarized above, the subject invention provides methods of evaluating a subject for a condition in a subject specific manner. In other words, the invention provides subject or patient customized methods of assessing a subject for a particular condition, where the methods are tailored for use with the subject being assayed. By condition is meant a physiologic condition or state of a subject. Condition is used broadly to refer not only to disease conditions, but also other physiological conditions that are not necessarily disease conditions, e.g., non-disease physiological conditions, such as metabolic states, other health-related conditions, etc.

The term “evaluate” is used herein broadly to refer not only to the diagnosis or detection of a given condition of interest, but also to the monitoring of a condition over a given period of time. As such, in certain embodiments one uses the subject methods to diagnose a subject for the presence of a given condition, i.e., to determine whether a subject has a given condition. In yet other embodiments, one uses the subject methods to monitor, predict, or track, i.e., watch or observe, the progression of a condition in a subject over a period of time.

The subject or patient evaluated in the subject methods may be a variety of different organisms, but is generally an animal, where animals of interest in many embodiments are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In many embodiments, the patients or subjects from which the sample is obtained in the subject methods will be humans.

As summarized above, the subject methods are array-based methods. By “array-based” is meant that the assay protocols of the subject invention employ an array (as defined above) to assay or test a given sample. As such, in the subject array-based assays, a sample is contacted with an array and binding complexes on the surface of the array are then detected to provide an assay result, as described in greater detail below.

A feature of the subject methods is that they are patient specific methods, where the methods include one or more patient specific array-based assays in which the features interrogated in the assays are those that have been previously identified in an initial or prior assay as relevant to the patient and condition. In other words, a characteristic of the subject assays is that a set of features previously identified as relevant (for the given patient and condition of interest) from a larger group of features in an initial feature identification array-based assay is interrogated or evaluated, i.e., tested or screened.

As such, certain embodiments of the subject methods may be viewed as including a first or initial general array-based assay that identifies the subject or patient specific subset of features for use in the subsequent patient specific assay(s), and then at least one additional array-based assay, where the at least one additional array-based assay differs from the first general array-based assay in that not all of the features of the array interrogated in the first array-based assay are interrogated. In other words, the methods of certain embodiments are characterized by performing at least first and second sequential array-based assays, where the second array-based assay differs from the first in that not all of the array features evaluated in the first assay are evaluated in the second assay.

A further characteristic of the subject methods is that those features evaluated or interrogated in the one or more additional array-based assays performed after the first or initial array-based assay include features, and are sometimes limited to features, that have been identified as relevant in the first or initial array-based assay with respect to the given host (i.e., subject or patient) and condition. In other words, the one or more additional array-based assays differ from the first or initial array-based assay by evaluating or interrogating a different collection of features than those interrogated in the first or initial array-based assay, where the different collection of features interrogated in the one or more additional array-based assays is a collection that includes features identified as relevant (with respect to the particular subject and condition) in the initial or first array-based assay.

As such, the first or initial array-based assay is a general assay in which a total number of features are interrogated to identify features for use in the one or more additional patient specific array-based assays. By general assay is meant an assay that is not tailored for a specific subject or patient, i.e., it is an assay which is not customized for a given subject or patient. As such, the initial general assay may be one that is general with respect to a species or a subpopulation thereof, e.g., the assay may be one that is species specific that has features for a large number of genes/proteins of a given species, or disease specific, that has features of genes/proteins that have been previously determined to be relevant for a particular disease or condition. However, in any event the first array-based assay of the subject methods is not one that is subject or patient specific, in that the features of the array interrogated or evaluated in the first array-based assay are ones that have not been previously identified as relevant for the specific patient and condition that is the subject of the patient specific array-based assay components of the subject methods, i.e., for which the assay is being performed.

In the first array-based assay of the subject methods of these embodiments, a total number of array features is evaluated or interrogated as described below, where the total number in many embodiments ranges from about 50 to about 100,000 or more, including from about 100 to about 50,000, such as from about 500 to about 10,000.

In the first or initial assay of the subject methods, a sample from the subject is assayed using an array-based assay to identify those features of the initial total number of features interrogated in the first or initial assay that are relevant for the particular subject and condition of interest. In other words, the first or initial array-based assay is performed to identify a subset (of the initially interrogated total number of features) that is made up of features that are relevant to the particular subject and condition being assayed. A given feature is relevant if its interrogation in an assay provides useful information with respect to the condition being evaluated. As such, features identified as relevant in the first or initial array-based assays of the subject methods are those features whose interrogation provides useful information in evaluation of the specific patient (subject/individual/host) for the specific condition of interest. The number of features making up the identified subset of the total number of features may vary, but in certain embodiments may be less than about 80% of the total number of features, including less than about 70%, 60%, 50%, 33%, 25%, 15%, 10%, 5%, etc. As such, performance of the first or initial array-based assay provides, i.e., identifies or selects, a subset of features that are known to be relevant to the particular subject and condition thereof being assayed in the one or more patient specific array-based assays.

Following the initial or first array-based assay, at least one or more additional patient specific array-based assays are performed, e.g., on a portion of the same sample as assayed in the first assay or on a new sample from the same subject. A feature of the one or more additional array-based assays is that they are subject or patient specific, i.e., they are tailored or customized for use with the particular subject or patient and for the particular condition being assayed.

As the array-based assays performed subsequent to the initial or first assay are subject or patient specific, as described above, they are characterized in that a collection of features that is different from the total number of features of the first array-based assay is evaluated or interrogated, where the collection of features that is interrogated is one that includes the subset of relevant features identified in the first or initial array-based assay. In the one or more additional array-based assays, the group or collection of features that is interrogated is one that at least includes the previously identified subset of features, and may include features other than the previously identified subset, so long as the collection of features is not the same as the total number of features of the first array-based assay.

The number of one or more additional patient specific array-based assays that is performed in a given method may vary greatly, where the number is at least one, such as least about 5, at least about 10, at least about 25, etc., where the number may be as great as 50, 100, 200 or more.

As mentioned above, the first and at least one additional array-based assays are sequential, such that the at least one additional array-based assay is performed at a time subsequent to the first or initial array-based assay. The period of time separating the first and at least one additional patient specific array-based assay, as well as any two subsequent additional array-based assays of an overall protocol, may vary, where in many embodiments it is at least about 1 min, including at least about 1 hr, at least about 24 hrs, at least about 1 week, at least about two weeks, at least about 1 month, etc., such that it may be at least within the same day, but may also be within weeks or months, and in certain embodiments within a year. In some embodiments the first and second array-based assay may be performed on a recurring basis, e.g., at least about every 24 hrs, at least about every 1 week, at least about every two weeks, at least about every month, at least about every year, etc. The manner in which the initially identified subset of features is interrogated or evaluated may vary, depending on the particular protocol being performed. In other words, the way in which patient specific array-based assays only interrogate the collection (e.g., subset) of features identified as relevant in the initial array-based assay may vary, depending on a number of factors, such as the nature of the array employed in the patient-specific array based assay.

As such, in certain embodiments one can employ an array that includes only those features making up the above-described collection of features, e.g., an array having only those features that make up the identified subset of features. Such an array can be obtained/prepared using any convenient protocol for fabricating the array, where representative suitable protocols are summarized above.

In yet other embodiments, one may use an array with more features than the subset/collection that is the subject of the one or more additional array-based assays (e.g., the array employed in the initial array-based assay), but only interrogate or evaluate those features making up the collection/subset of interest, e.g., by employing a feature evaluation program/algorithm that masks out non-relevant features and therefore only interrogates features of the collection/subset of interest (e.g., as described in U.S. patent application Ser. No. ______ (Attorney Docket No. 10021296-1), entitled “Masking Chemical Arrays” and filed on Jul. 31, 2003, the disclosure of which is herein incorporated by reference), by employing a protocol that generates labeled target molecules (e.g., by employing gene-specific primers) for only those features of interest (providing higher sensitivity than other protocols and therefore allowing one to assay smaller patient samples); etc. In certain embodiments, the patient specific array-based assays may be run in conjunction with a general array-based assay, such as the original assay used to identify the relevant features of interest, depending on the particular clinical assay protocol being followed.

In the subject methods, each array-based assay is characterized by assaying a sample from the subject of interest with an array, as summarized above. The sample employed in the initial and one or more additional patient specific array-based assays may be the same or different. The sample may be any of a variety of different physiological samples that are obtainable from a patient or subject, where representative samples of interest include, but are not limited to: whole blood, plasma, serum, semen, saliva, tears, urine, fecal material, sweat, buccal fluid, skin fluid, spinal fluid and hair; in vitro cell cultures, including a growth medium, cells and cell components, tissue biopsies and samples,-surgically-excised tissues, and the like. The sample may or may not be pretreated, e.g., by freezing or the addition of one or more agents of interest, such as preservatives, chaotropic agents, labeling agents, etc., as is known in the art.

In certain embodiments the assays are clinical assays, which means that the assays are conducted to provide information on the current or future health or condition, diagnosis, treatment (including determination of treatment regimen), prevention, and/or monitoring of a condition of the patient or subject of interest. In other words, the assays are conducted to detect the presence of and/or determine the stage of, severity of, etc., a condition of the patient. The condition may or may not be a disease condition. As such, in certain embodiments, the clinical assay is performed to diagnose the presence of, and/or determine the stage or monitor the progression of, a disease condition. In other embodiments, the assay is performed to determine the appropriate treatment regimen for the patient. In yet other embodiments, the condition may not be a disease condition, but merely a propensity or predisposition for a disease condition. In yet other embodiments, the condition may not be a classical disease condition, but merely a physiological state that can be detected and/or monitored by array-based assay, e.g., a metabolic rate determination, etc.

The nature of the array-based assays may vary, where the assays may be: genomic assays, in which nucleic acid targets in the sample are hybridized to an array of nucleic acid probes on the array; proteomic assays, in which proteinaceous analytes in the sample are specifically bound to an array of binding agent probes on the array; or other types of array assays using other types of arrays, usually biopolymeric arrays, to detect the presence of one or more analytes of interest in the sample. Arrays of interest include those described above.

In general, assay protocols performed according to the subject methods include the following steps: (1) sample obtainment and (2) assay of the sample. In the first sample obtainment step, a sufficient amount of sample is obtained from the patient or subject of interest. In many embodiments, the sample is a fluid sample, where the volume of sample obtained in this step may range from, in fluid volumes, from about a few pL (equaling one or several cells) to about 10 mL (as in a blood sample from a human) to much larger quantities such as blood or urine samples from horse or other large animals, and in the case of tissue samples, from about 1-10 cells (or pgs tissue) to about 10⁶ or 10⁷ cells (ng μgs tissue) in certain embodiments. The sample is typically obtained and placed in a sample containment means, in which it may then optionally be stored for a period of time in the optional sample storage step. The period of time during which the sample is stored in this step may vary, where the sample is stored typically for at least about several minutes to about 30 minutes or more, but may frequently be overnight such as at least about a week, where the period of time during which the sample is stored may be as long as a year or longer, such as years, decades or longer, where in certain embodiments the sample may be transported or moved from a first location to a second location.

Following sample obtainment, the array-based assay component of the subject methods is performed, in which the presence of a particular analyte(s) in a given sample is detected at least qualitatively, if not quantitatively. Protocols for carrying out such assays with arrays are well known to those of skill in the art and need not be described in great detail here. Generally, the sample is contacted with an array under conditions sufficient for the analyte(s) (if present) to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system, e.g., an isotopic or fluorescent label present on the analyte, etc. The presence of the analyte in the sample is then deduced from the detection of binding complexes on the substrate surface.

Specific clinical array-based assay applications of interest include hybridization assays in which a nucleic acid array is employed. In these assays, a clinical sample is first obtained and then prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assay protocols that may be employed in a given clinical array based assay include: simple contact of an individual sample with an array; differential gene expression analysis assays where the sample is co-hybridized and compared to a reference; and the like. Patents and patent applications describing methods of using nucleic acid arrays in various applications, including clinical array diagnostic applications, include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Patents and patent applications describing methods of using proteomic arrays in various applications, including clinical array diagnostic applications, include: U.S. Pat. Nos. 4,591,570; 5,171,695; 5,436,170; 5,486,452; 5,532,128; and 6,197,599; the disclosures of which are herein incorporated by reference; as well as published PCT application Nos. WO 99/39210; WO 00/04832; WO 00/04389; WO 00/04390; WO 00/54046; WO 00/63701; WO 01/14425; and WO 01/40803; the disclosures of the United States priority documents of which are herein incorporated by reference.

In certain embodiments, the subject methods include a step of transmitting data from at least one of the quality evaluation and clinical assay steps, as described above. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in practicing the methods of the subject invention, the array will typically be exposed to a clinical sample (for example, a clinical sample-that has been fluorescently labeled) and the array then read. Reading of the array may be accomplished by illuminating the array and reading the location and intensity of resulting fluorescence at each feature of the array to detect any binding complexes on the surface of the array. For example, a scanner may be used for this purpose, such as the AGILENT MICROARRAY SCANNER device available from Agilent Technologies, Palo Alto, Calif. Other suitable apparatuses and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

While the above methods section is described in terms of an overall method of performing a first or initial array-based assay to identify a collection/subset of features relevant to a specific patient and condition of interest and then performing one or more additional patient specific array-based assays in which only the previously identified collection of features is interrogated, the above description also provides full support for those embodiments of the present invention that are directed to methods of identifying a collection of features for use in patient specific array-based assays, method of producing patient specific arrays for use in such assays and methods of performing patient specific array-based assays characterized by interrogating features previously identified to be relevant to a particular patient and condition of interest.

Utility

The subject invention finds use in array-based assays, and particularly clinical array-based assays. As indicated above, the clinical array-based assays to which the sample is subjected in the subject methods may be diagnostic assays, e.g., where the presence of a certain condition, such as a disease condition, is determined; or part of a therapeutic regimen, e.g., to monitor the progression of the disease condition.

The subject methods may be used to detect/monitor any condition whose presence and/or state is associated with a defined biopolymeric, e.g., genomic or proteomic, profile, such that a determined biopolymeric profile can be used to determine the presence or state of the condition of interest. A variety of conditions may be detected and/or monitored according to the subject invention. Representative conditions that are amenable to detection and/or monitoring using array-based assays include, but are not limited to: neoplastic disease conditions, cardiovascular disease conditions, pathogenic disease conditions (such as viral disease conditions), neurological, immune function and the like.

One specific representative application of interest is the detection of residual disease in a subject, such as the detection of residual disease in cancer patients by array-based protocols, particularly array-based comparative genomic hybridization (CGH). One challenge in such applications is that they require optimal sensitivity in terms of the ability to detect a small population of residual cancer cells amongst a larger population of different normal cells. In addition to the sensitivity requirement, assays designed to detect residual disease need to be cost effective such that a patient can be monitored multiple times. Tailoring an array-based CGH assay to detect only the genetic alterations (or a subset thereof) found to be relevant in a specific patient according to the present methods enables more cost-effective and sensitive subsequent or secondary patient assays. The increased sensitivity results from one or more of: 1) use of smaller microarrays which requires less sample for analysis; 2) the ability to target a small and known-to-be-relevant number of targets which provides the opportunity to employ target (e.g., PCR preparation) and/or signal (e.g., sandwich assay) amplification protocols; and 3) the ability to target a small and known to be relevant number of targets which provides the opportunity to employ more robust feature extraction/interpretation algorithms.

Yet another specific representative application of interest is monitoring disease progression in individuals, e.g., monitoring disease progression in individuals with an elevated risk of developing cancer. One of the early events in the development of many cancers (e.g., lung, bladder, esophageal, head and neck, etc.) is the initial localized appearance of an advanced neoplastic clone. These early clones typically have combinations of alterations that are tissue and patient specific. Over time these premalignant clones may undergo extensive expansion before subsequently developing into an invasive carcinoma. Patients with symptomatic advanced premalignant lesions (e.g., smokers with heavy coughs, patients with Barrett's esophagus) are often placed in surveillance where multiple biopsies are acquired over varying periods of time to monitor for signs of expansion and invasion. According to the present invention, these materials are screened for genomic alterations or changes in RNA levels with comprehensive arrays as a baseline measurement, e.g., in a first or initial array-based assay according to the subject methods. These initial assays identify patients with one or more localized neoplastic clones and then provide a unique set of markers, such as a series of genomic alterations, specific to each patient that are used in follow up visits to the clinic, e.g., in patient-specific array-based assays produced according to the subject invention. The product patient-specific array based assays can also be used to monitor the efficacy of interventions, such as chemoprevention and ablation therapies, on the disease.

Yet another example of an application of the present methods is a protocol having an initial generic gene expression array that contains signal transduction pathways, immune system elements, multiple disease-specific-gene sets. Based upon the first “screen,” a tailored array for that patient's specific condition is produced and used for subsequent diagnostic analysis in patient specific array-based assays, as well as for long-term disease/condition/syndrome monitoring.

Kits

Kits for use in array-based assays according to the present invention are also provided. The kits at least include one or more components employed in either an initial or patient specific array-based assay, as described above. As such, in certain embodiments, the kits may include an initial general array, and instructions for using the array to identify a collection of patient specific/condition relevant features. In yet other embodiments, the kits may include an array, and an element for restricting interrogation of the array to previously identified patient specific/condition relevant features. Such feature restriction interrogation elements may include a feature extraction algorithm recorded on a computer readable medium, one or more reagents for generating labeled targets exclusively to features of interest, etc. In yet other embodiments, the kits may include an array that only displays previously identified patient specific/condition relevant features. The kits may further include one or more additional components necessary for carrying out the array-based clinical assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay. Such kits also typically include instructions for use in practicing array-based assays.

As mentioned above, the kits may also include a computer readable medium including programming, as discussed above, and instructions. Programming according to the present invention can be recorded on computer readable media, e.g., any medium that can be read and accessed directly or indirectly by a computer. Such media include, but are not limited to: magnetic tape; optical storage such as CD-ROM and DVD; electrical storage media such as RAM and ROM; and hybrids of these categories such as magnetic/optical storage media. One of skill in the art can readily appreciate how any of the presently known computer readable mediums can be used to create a manufacture that includes a recording of the present programming/algorithms for carrying out the above-described methodology. In certain embodiments, the programming is further characterized in that it provides a user interface, where the user interface presents to a user the option of selecting among one or more different, including multiple different, quality criteria, etc. The instructions may include installation or setup directions. The instructions may include directions for use of the invention.

In addition, the kits will typically include instructions for using the kit components in a method according to the present invention. The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

It is evident from the above discussion that the above-described invention provides a number of advantages to the field of array-based assay, and particularly clinical assays. For example, by using the subject methods one can develop patient specific customized assays from an individual array design, providing significant resource savings. In addition, one can develop patient specific assays that can achieve higher sensitivity than can be obtained with general non-patient specific, e.g., population specific, array-based assays. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of evaluating a subject for a condition, said method comprising:
(a) performing a first array-based assay on a sample from said subject with an array having a total number of features to identify a subset of said total number of features relevant to said condition and said subject; and
(b) performing at least a second array-based assay on a sample from said subject, where a collection of features different from said total number of features and including said subset of features is interrogated;
to evaluate said subject for said condition.

2. The method according to claim 1, wherein said condition is a disease or health-related condition.

3. The method according to claim 1, wherein said first and at least second array-based assays are performed sequentially.

4. The method according to claim 1, wherein arrays displaying the same total number of features are employed in said first and at least second array-based assays.

5. The method according to claim 4, wherein said at least second array-based assay comprises employing an algorithm that masks out results from features of said total number that are not members of said subset.

6. The method according to claim 4, wherein said at least second array-based assay comprises employing a protocol that produces labeled targets for only those feature members of said subset.

7. The method according to claim 1, wherein said second array-based assay employs a second array that includes a different number of features from said total number of features, wherein said different number of features includes said subset of features.

8. The method according to claim 7, wherein said second array employed in at least said second array-based assay includes only said subset of features.

9. The method according to claim 1, wherein said features comprise nucleic acids.

10. The method according to claim 1, wherein said features comprise peptides.

11. The method according to claim 1, wherein said method is a method of diagnosing said subject for said condition.

12. The method according to claim 1, wherein said method is a method of monitoring said subject for said condition.

13. The method according to claim 1, wherein said subject is a human.

14. A method of identifying a collection of target features for use in a subject specific array-based assay, said method comprising:
(a) performing a first array-based assay on a sample from a subject with an array having a total number of features to identify a subset of said total number of features relevant to said condition and said subject; and
(b) identifying a collection of features different from said total number that includes said subset to identify said collection of target features for use in a subject specific array-based assay.

15. The method according to claim 14, wherein said subject specific array-based assay evaluates said subject for a health related or disease condition.

16. The method according to claim 14, wherein said features comprise nucleic acids.

17. The method according to claim 14, wherein said features comprise peptides.

18. The method according to claim 15, wherein said assay is a method of diagnosing said subject for said health-related or disease condition.

19. The method according to claim 15, wherein said assay is a method of monitoring said subject for said health related or disease condition.

20. The method according to claim 14, wherein said subject is a human.

21. A method of producing an array for use in a subject specific array-based condition evaluation protocol, said method comprising:
(a) performing a first array-based assay on a sample from a subject with an array having a total number of features to identify a subset of said total number of features relevant to said condition and said subject; and
(b) producing a second array comprising a collection of features different from said total number that includes said subset;
to produce said array for use in a subject specific array-based condition evaluation protocol.

22. The method according to claim 21, wherein said condition is a health-related or disease condition.

23. The method according to claim 21, wherein said features comprise nucleic acids.

24. The method according to claim 21, wherein said features comprise peptides.

25. The method according to claim 21, wherein said evaluation protocol is a method of diagnosing said subject for said condition.

26. The method according to claim 21, wherein said evaluation protocol is a method of monitoring said subject for said condition.

27. The method according to claim 21, wherein said subject is a human.

28. An array produced according to the method of claim 21.

29. The array according to claim 28, wherein said array is a nucleic acid array.

30. The array according to claim 28, wherein said array is a peptide array.

31. A kit comprising:
(a) an array comprising a total number of features; and
(b) a feature interrogation restriction element for restricting interrogation of said total number of features to features in a subset of said total number that are known to be relevant to a specific subject and a condition thereof.

32. The kit according to claim 31, wherein said element is an algorithm recorded on a computer readable medium.

33. The kit according to claim 31, wherein said element comprises one or more reagents for generating labeled target analytes from a patient sample.

34. A kit comprising:
(a) an array according to claim 28; and
(b) instructions for using said array in an array-based subject condition evaluation protocol.

35. A method of performing a subject specific array-based condition evaluation assay, said method comprising:
conducting an array-based assay on a sample from a subject, wherein said array-based assay interrogates features of an array previously identified in a prior array-based assay as relevant to said subject and said condition.

36. The method according to claim 35, wherein said array-based assay comprises using an array that includes features not previously identified in a prior array-based assay as relevant to said subject and said condition.

37. The method according to claim 35, wherein said array-based assay comprises:
(a) contacting a sample from said subject with an array according to claim 28; and
(b) reading said array to detect any binding complexes on the surface of the said array to obtain an assay result.

38. The method according to claim 35, wherein said method further comprises a data transmission step in which a result from said array-based assay is transmitted from a first location to a second location.

39. A method according to claim 38, wherein said second location is a remote location.

40. A method comprising receiving data representing a result of a reading obtained by the method of claim 35.
